(19) United States
(12) Patent Application Publication
(10) Pub. No.: US 2011/0244595 A1
(43) Pub. Date: Oct. 6, 2011

(54) BIOMEDICAL CHIP FOR BLOOD COAGULATION TEST, METHOD OF PRODUCTION AND USE THEREOF

(75) Inventors: Chen-Kuei Chung, Tainan (TW); Hsien-Chang Chang, Tainan (TW); Chia-Chern Chen, Tainan (TW); Yu-Sheng Chen, Tainan (TW); Cheng-Ting Li, Kaohsiung (TW)

(73) Assignee: National Cheng Kung University, Rosemead, CA (US)

(21) Appl. No.: 13/073,834

(22) Filed: Mar. 28, 2011

(30) Foreign Application Priority Data
Apr. 1, 2010 (TW) 99110111

(51) Int. Cl. G01N 33/48; B23P 11/00

(52) U.S. Cl. 436/501; 422/73; 137/15.01

(57) ABSTRACT

In a biomedical chip for blood coagulation tests and its manufacturing method and use, the biomedical chip comprises a substrate layer, a middle layer, and a cap layer, engaged and stacked with each other to define a microfluidic channel which has a first inlet and an outlet of the microfluidic channel respectively. A mixing interval is expanded outward from the microfluidic channel and interconnected to a second inlet, and has an interconnect portion and a capillary portion disposed between the substrate layer and the cap layer, and more specifically disposed around the periphery of the interconnect portion. With the biomedical chip having the substrate layer and cap layer made of a hydrophilic material, the blood and the reagent can be driven automatically by the capillary force of the microfluidic channel to flow and mix with each other, and the hydrophilic capillary force can be permanently maintained.

### RELATED APPLICATIONS

The present invention claims priority to Taiwanese Patent Application Serial Number 99110111, filed on Apr. 1, 2010, the entirety of which is hereby incorporated by reference.

### BACKGROUND OF THE INVENTION

1. Field of the Invention

The present invention relates to a biomedical chip, and more particularly to a biomedical chip for blood coagulation tests, which is adapted for mixing and transporting at least two types of reagents.

2. Description of Related Art

Conventional blood coagulation tests are conducted in laboratories, and usually take time to obtain a plasma sample that requires blood centrifugation of the sample and very expansive and large instruments operated by professionals. Therefore, the conventional blood coagulation tests are not suitable for an immediate use by the general public and relatively troublesome for medical professionals who need to know experimental results immediately. Although some models of small instruments related to the blood coagulation tests are available in the market, these instruments are generally restricted to patients who take anticoagulants, not for the general public.

In recent years, the technology of biomedical testing chips is mature and can be used for blood coagulation tests. One of the major development trends of the biomedical testing chips is to drive and mix microfluids, particularly to overcome the difficulty of driving a highly viscous microfluid (such as blood). At present stage, an external micropump is added to drive microfluid, but the way of driving microfluids by the micropump requires a larger device as well as a higher cost, and the structural design of the chip is more difficult and unfavorable for manufacturing. Therefore, manufacturers try to reduce the manufacturing cost of the micropump by using surface tension to drive the highly viscous microfluid instead.

In the chip of driving the microfluid by surface tension, if a microfluidic channel has a highly hydrophilic surface, a large driving force of the microfluid can be generated. At present stage, a polymer material (such as SU8 or PDMS) is generally used for manufacturing micro-channel for microfluidic self-driven chips, and an oxygen plasma treatment or another surface modification treatment is used for changing the material surface from a hydrophobic surface to a highly hydrophilic and moist surface to achieve the goal of self-driving and transporting. However, if using the oxygen plasma and thermal treatments are the only ways to improve the hydrophilic property of PDMS, the highly hydrophilic property will disappear after about tens of minutes. If a chemical dip treatment is performed to the PDMS after the oxygen plasma treatment, the hydrophilic property can remain for several days but the problem of returning to the hydrophobic surface still exists.

Besides the polymer materials including PDMS and SU8, glass is also a common material used for manufacturing a microfluidic chip, but conventional manufacturing and bonding processes of the glass chip have drawbacks including a high level of complexity and a high thermal bonding temperature.

### SUMMARY OF THE INVENTION

Therefore, it is a primary objective of the present invention to provide a microfluidic self-driven biomedical chip having a permanent hydrophilic capillary force for blood coagulation tests.

Another objective of the present invention is to provide a biomedical chip for blood coagulation tests with a permanent hydrophilic capillary force.

A further objective of the present invention is to provide a biomedical chip for blood coagulation tests and for driving and mixing at least two types of reagents automatically.

To achieve the foregoing objectives, the present invention provides a biomedical chip for blood coagulation tests, comprising: a substrate layer made of a hydrophilic material, a middle layer, and a cap layer made of a hydrophilic material, sequentially engaged and stacked from bottom to top with each other, wherein the substrate layer, the middle layer and the cap layer define a microfluidic channel formed at the cap layer, having a first inlet and an outlet at two opposite ends of the microfluidic channel respectively; a second inlet being disposed proximate to the first inlet and interconnected to the microfluidic channel; and a mixing interval expanded radially outward and interconnected to the second inlet and the externally expanded mixing interval having a diameter greater than the diameter of the second inlet, wherein an interconnect portion is disposed at the bottom of the second inlet; and a capillary portion is disposed between the substrate layer and the cap layer, and interconnected to the interconnect portion and disposed around the periphery thereof, wherein the microfluidic channel's internal diameter is small enough to produce a capillary force to drive blood in the first inlet and a reagent in the second inlet to flow along a lengthwise direction, such that the blood is driven automatically to pass through the capillary portion to absorb the reagent in the interconnect portion and flow towards the outlet.

To achieve the foregoing objectives, the present invention further provides a method of manufacturing a biomedical chip for blood coagulation tests in accordance with the present invention, and the method comprises the steps of: (a) attaching and fixing a middle layer onto the top of a hydrophilic substrate layer; (b) forming a slender penetrating microchannel at the top of the middle layer, wherein the microchannel has an externally expanded section radially expanded outward; (c) forming a first inlet hole, a second inlet hole and a outlet hole with an interval apart from each other and passed through the hydrophilic cap layer by a laser manufacturing method, and the second inlet hole having a diameter smaller than the diameter of the externally expanded section; and (d) stacking and fixing the cap layer produced in Step (c) onto the top side of the middle layer to cover the microchannel, such that the cap layer, the substrate layer and the middle layer define an internal diameter of the microchannel which is small enough to produce a capillary force for driving the blood to flow in the microfluidic channel automatically, and the first inlet hole and the outlet hole are interconnected to both opposite ends of the microfluidic channel respectively, and the second inlet hole and the externally expanded section are interconnected correspondingly.

To achieve the foregoing objectives, the present invention uses the aforementioned biomedical chip for blood coagulation tests for driving and mixing two types of reagents, and the use comprises the steps of: (a) dropping a first reagent into the first inlet, and using the capillary force produced by the microfluidic channel to drive the first reagent to flow automatically; and (b) adding a second reagent to be mixed into the interconnect portion from the second inlet after the first reagent is passed along the capillary portion of the mixing interval, bypassed through the interconnect portion of the mixing interval, and passed through the mixing interval, such that after the second reagent is sucked into the capillary portion by the capillary force of the capillary portion and mixed with the first reagent, the first and second reagents are passed and mixed through the mixing interval.

To achieve the foregoing objectives, the present invention uses the aforementioned biomedical chip for blood coagulation tests for driving and mixing two types of reagents, and the use comprises the steps of: (a) coating a dry powdered second reagent onto an internal wall of the mixing interval; and (b) dropping a liquid first reagent into the first inlet, and using the capillary force produced by the microfluidic channel to drive the first reagent to flow automatically, such that the first reagent passing through the capillary portion gradually carries away the dry powdered second reagent in the mixing space, and after the second reagent is mixed with the first reagent, the first and second reagents are passed through the mixing interval.

Therefore, the present invention can achieve the following effects. The hydrophilic material is used for making the substrate layer and the cap layer to complete the biomedical chip for blood coagulation tests, and the hydrophilic capillary force of the microfluidic channel is used for driving the blood with a high viscosity to flow automatically, as well as driving and mixing the two types of liquids and maintaining their hydrophilic property permanently.

### BRIEF DESCRIPTION OF THE DRAWINGS

FIG. 1 is an exploded view of a biomedical chip for blood coagulation tests in accordance with a first preferred embodiment of the present invention;

FIG. 2 is a top view of the preferred embodiment of the present invention;

FIG. 3 is a schematic view of performing a water-assisted laser manufacturing process to a cap layer in accordance with the preferred embodiment of the present invention;

FIG. 4 is a schematic view similar to FIG. 3 and illustrating the manufacture of a microchannel by attaching a middle layer for carrying the top side of the substrate layer by a water-assisted laser manufacturing process; and

FIG. 5 is a histogram of time for performing each coagulation time test of citrated blood in accordance with the preferred embodiment of the present invention.

### DETAILED DESCRIPTION OF THE PREFERRED EMBODIMENTS

The technical contents, characteristics, and effects of the present invention will become apparent with the detailed description of preferred embodiments together with related drawings as follows.

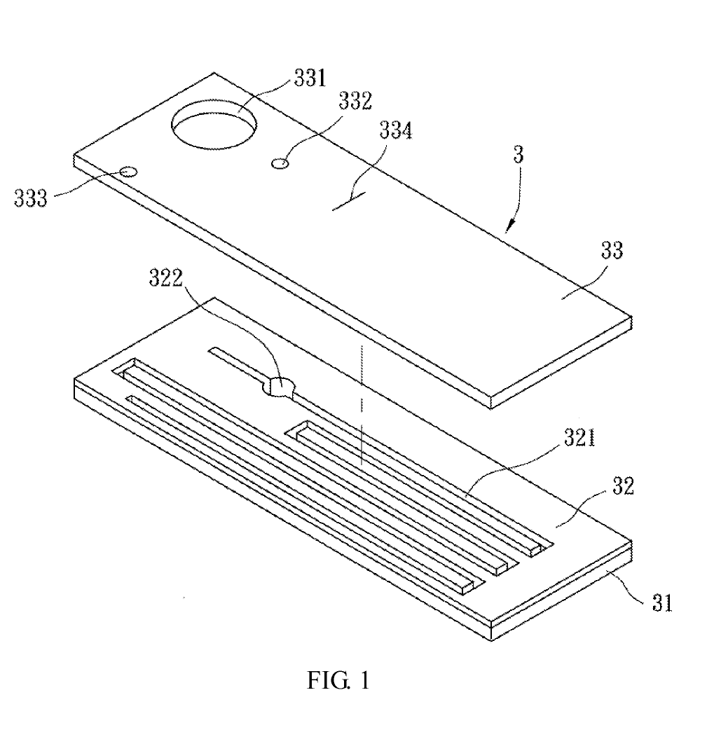

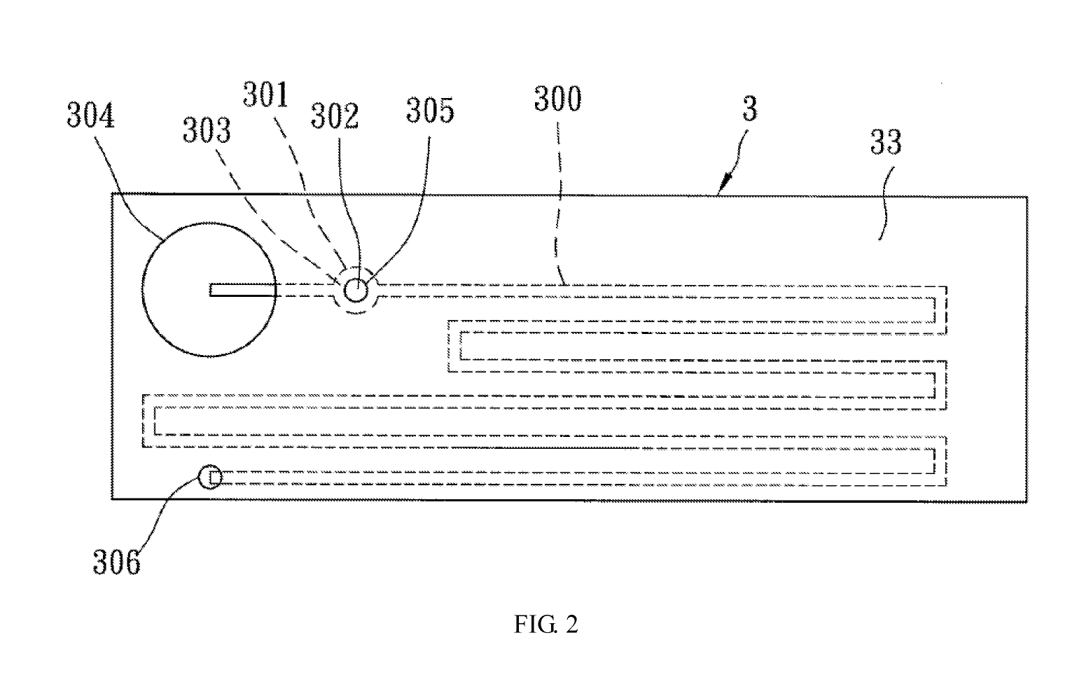

With reference to FIGS. 1 and 2 for a biomedical chip 3 for blood coagulation tests in accordance with a preferred embodiment of the present invention, the biomedical chip 3 is applied for performing a coagulation time test for whole blood and transporting and mixing two types of reagents automatically, wherein the reagents can be dry powdered reagents or liquid reagents. The biomedical chip 3 comprises a substrate layer 31, a middle layer 32 and a cap layer 33 sequentially stacked from bottom to top and engaged with each other.

The substrate layer 31 and the cap layer 33 are made of hydrophilic materials. In this preferred embodiment, the substrate layer 31 and cap layer 33 are made of hydrophilic glass, wherein, a first inlet hole 331, a second inlet hole 332 and a outlet hole 333 are formed at the top side of the cap layer 33 and with an interval apart from each other, and a marked line 334 is extended from the front to the rear and disposed at a position with a predetermined distance from the right side of second inlet hole 332 at the top side of the cap layer 33.

The middle layer 32 is a double-sided adhesive tape having a slender microchannel 321 penetrated from top to bottom and bent reciprocally from left to right and extended from front to rear, and a circular externally expanded section 322 expanded radially outward from a position proximate to the left side of the microchannel 321, and the externally expanded section 322 has a diameter greater than the diameter of the second inlet hole 332.

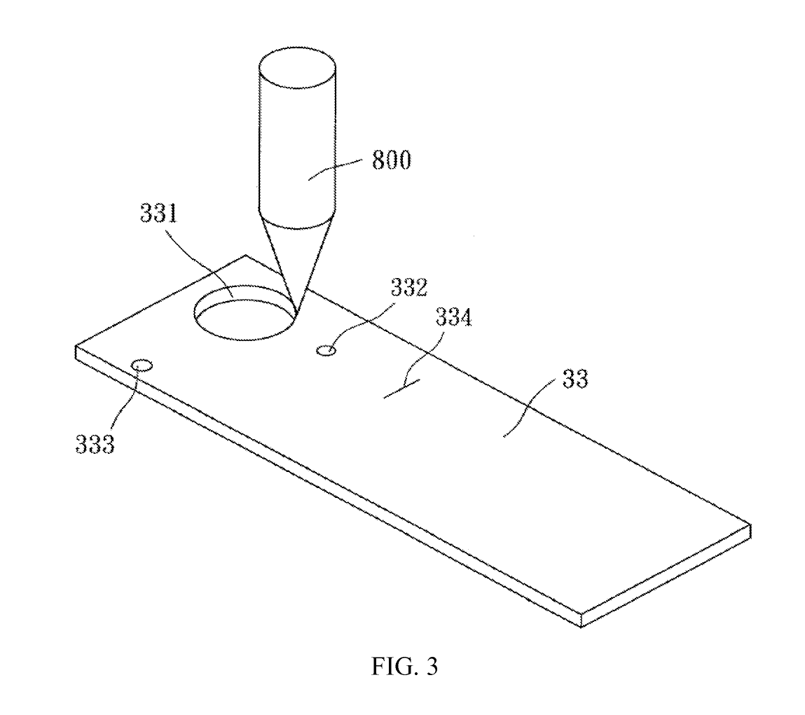

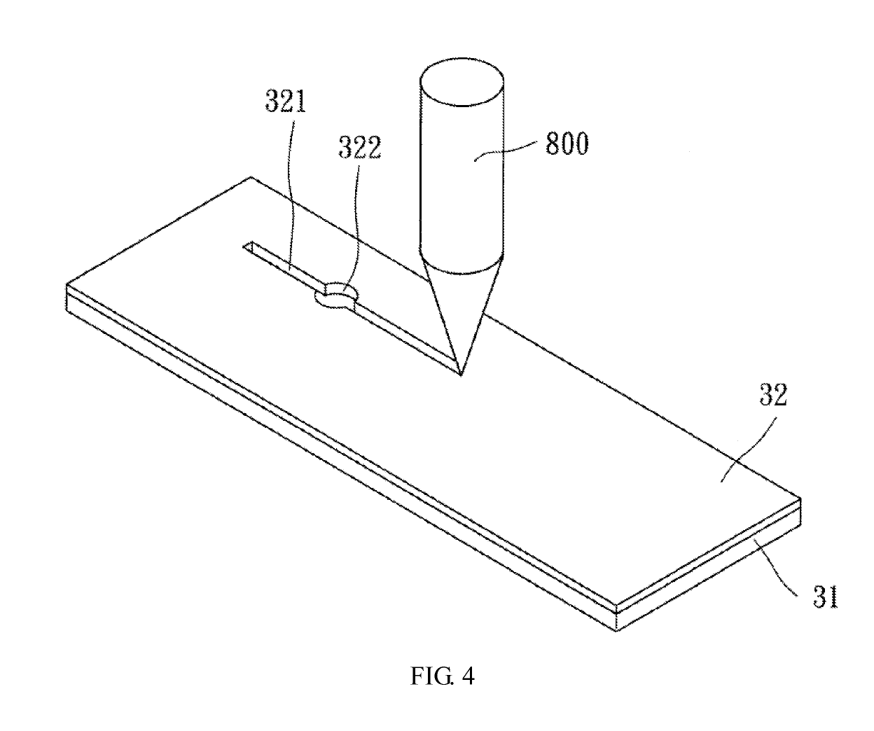

In FIGS. 3 and 4, the biomedical chip 3 is manufactured by a water-assisted laser equipment 800, wherein the first inlet hole 331, the second inlet hole 332 and the outlet hole 333 are formed on the cap layer 33. Before the middle layer 32 is produced, it is necessary to adhere and fix the cap layer 22 to the top of the substrate layer 31. During the process of adhering and fixing the cap layer 22 onto the top of the substrate layer 31, the water-assisted laser manufacturing method is used for forming the microchannel 321 at the top of the middle layer 32.

The biomedical chip can then be engaged and stacked with each other. Since the middle layer 32 of this preferred embodiment is a double-sided adhesive tape with the adhesive property, the cap layer 33 can be stacked and coupled to the top of the middle layer 32 directly, and the microchannel 321 can be sealed. Now, the substrate layer 31, the cap layer 33 and the middle layer 32 define a continuously bent and extended microfluidic channel 300 which is interconnected to a first inlet 304 and an outlet 306 at opposite ends of the microfluidic channel 300, and disposed proximate to the second inlet 305 of the first inlet 304. The microfluidic channel 300 has a mixing interval 301 interconnected to the second inlet 305 and extended radially outward, and the mixing interval 301 has a diameter greater than the diameter of the second inlet 305, an interconnect portion 302 interconnected to and disposed precisely below the second inlet 305, and a circular capillary portion 303 disposed between the opposite lateral sides of the substrate layer 31 and the cap layer 33 respectively, and interconnected around the periphery of the interconnect portion 302.

In a further embodiment, the first inlet 304 has a diameter of 11 mm and the second inlet 305 has a diameter of 2 mm, while the outlet 306 has a diameter of 2 mm and a slender part of the microfluidic channel 300 has a width of 0.8 mm, and a depth of 175 μm. The mixing interval 301 of the microfluidic channel 300 has a diameter of 4 mm. Since the substrate layer 31 and the cap layer 33 are made of a hydrophilic material such as glass, the microfluidic channel 300 can produce a hydrophilic capillary force to drive the blood in the first inlet 304 and the reagent in the second inlet 305 to flow automatically. In other words, when the blood is filled into the first inlet 304, the capillary force of the microfluidic channel 300 will suck the blood automatically and drive the blood to pass through the capillary portion 303 of the mixing interval 301 automatically and flow towards the outlet 306. However, the microfluidic channel 300 and the mixing interval 301 of the present invention are not limited to the size as given above, but they can be adjusted according to actual usages and requirements.

The method of performing a blood coagulation test in accordance with another embodiment is described below. In this embodiment, whole blood (4.5 mL) extracted from a human body is mixed with 0.129 M sodium citrate (anticoagulant), wherein the ratio of blood to anticoagulant is equal to 9:1, so that the whole blood loses its coagulation effect to produce citrated blood. Three types of blood coagulation tests (vs. time) are preformed, including a recalcified coagulation time test by adding calcium ions, a recalcified coagulation time test by adding calcium ions and heparin, a recalcified coagulation time test by adding calcium ions and kaolin as described below.

(1) The recalcified coagulation time test by adding calcium ions comprises the following steps:

Step (1): Drop 150 μL of citrated blood into the first inlet 304, such that the citrated blood is driven by a capillary force of the microfluidic channel 300 to flow along the microfluidic channel 300 automatically.

Step (2): Drop 5 μL of calcium chloride solution into the second inlet 305 to start the recalcified coagulation time test and start counting the time, when the citrated blood passes through the mixing interval 301 and flows to a position of a marked line 334 at 20 mm downstream from the mixing interval 301. Now, the calcium chloride solution added into the interconnect portion 302 of the mixing interval 301 will be sucked gradually into the capillary portion 303 by the capillary force of the circular capillary portion 303 and mixed with the citrated blood, and then continued to flow through the mixing interval 301. In this embodiment, the concentrations of calcium chloride solutions used for the test are 1M and 3M respectively, and the calcium ions contained in these calcium chloride solutions are much greater than the concentration of calcium ions required for the coagulation of the whole blood.

Step (3): Stop counting the time when the blood in the microfluidic channel 300 is coagulated and remained still. The time period from adding the calcium chloride into the blood to coagulating the blood completely is defined as the blood recalcified coagulation time.

(2) The recalcified coagulation time test by adding calcium ions and heparin comprises the following steps:

Mix 150 μL of citrated blood and 0.1 ml of heparin (with a concentration of 5000 i.u./mL) uniformly before the test takes place, and then drop the blood mixed with the heparin into the first inlet 304. Drop 5 μL of 3 M calcium chloride solution when the blood flows to a position of a marked line 334 at 20 mm downstream from the mixing interval 301, and then start counting the time. Stop counting the time when the blood in the microfluidic channel 300 is coagulated and remained still. The time period from adding the calcium chloride into the blood to coagulating the blood completely is defined as the blood recalcified coagulation time.

(3) The recalcified coagulation time test by adding calcium ions and kaolin comprises the following steps:

Mix the citrated blood with kaolin in the test, and then drop the mixed blood into the first inlet 304, and drop calcium chloride solution into the second inlet 305 to start the test when the blood flows to a position of a marked line 334 at the downstream of the mixing interval 301, and stop counting the time when the blood in the microfluidic channel 300 is coagulated and remained still. The time period from adding the calcium chloride into the blood to coagulating the blood completely is defined as the blood recalcified coagulation time. In the mixing ratio of citrated blood to kaolin: 1.0 mL of citrated blood is added into 0.2 mg of kaolin, and the mixing concentration is approximately equal 0.2 mg/mL. The concentration of calcium chloride solution is equal to 3M, and the volume of calcium chloride solution is equal to 5 μL.

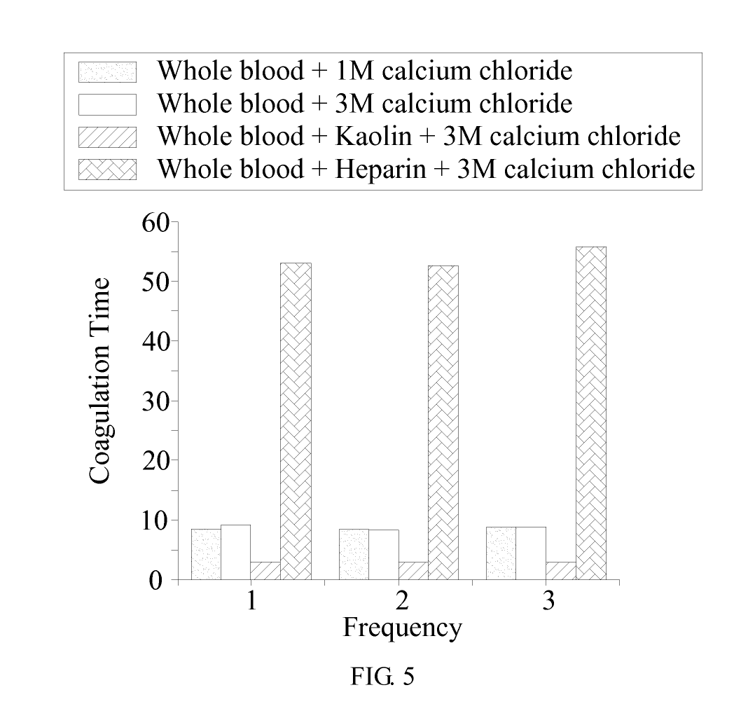

Referring to Table 1 shown below for the reference values adopted by clinical laboratories, and FIG. 5 for the coagulation time measured from the aforementioned three coagulation tests, the test results show that the concentration of calcium ions added into the citrated blood has no significant effect on the coagulation time.

TABLE 1

| Types of Blood Coagulation Tests | Coagulation time |
|---|---|
| Whole blood | 4-8 minutes |
| Citrated blood | Not coagulated |
| Citrated blood + Calcium ion | 5-12 minutes |
| Citrated blood + Heparin + | Depending on the concentration of |
| Calcium ion | heparin, the coagulation time can be |
| | extended up to many times. |
| Citrated blood + Kaolin + | Depending on the concentration of |
| Calcium ion | kaolin, the coagulation time can be |
| | shortened to less than half |

In the foregoing experiments, the coagulation time measured in the test of adding heparin is equal to 53.81±2.06 minutes, which is 5˜12 minutes longer than the normal coagulation time 5-12 measured in the test of simply adding calcium ions, and the recalcified coagulation time measured in the test of adding kaolin is reduced to half of the normal coagulation time, and all of the blood coagulation times measured in each blood coagulation test fall within the range of reference value adopted by the clinical laboratories. Therefore, the biomedical chip for blood coagulation tests in the present invention can actually be used for the blood coagulation tests.

From the aforementioned three tests, the structural design of the microfluidic channel 300 formed by the hydrophilic substrate layer 31 and the cap layer 33 of the biomedical chip 3 in the present invention can use the hydrophilic capillary force to drive highly viscous liquid, such that the blood can be driven without using any pump components, and the usage of the chip becomes easier and more convenient.

In addition to the use for the aforementioned blood coagulation tests, the biomedical chip for blood coagulation tests 3 of the present invention also can be used for driving, mixing and transporting other liquid reagents. When two types of liquid reagents are mixed, the first reagent is dropped into the first inlet 304 first, such that the first reagent is driven by the capillary force of the microfluidic channel 300 to flow towards the outlet 306 of the microfluidic channel 300. When the first reagent flows through the mixing interval 301, the second reagent is filled into the second inlet 305. Now, the second reagent at the interconnect portion 301 is gradually sucked by the capillary force of the capillary portion 302 and started to mix with the first reagent, and the mixed first and second regents can flow out of the mixing interval 301 to complete the mixing operation of the two types of reagents.

In the aforementioned mixing operation of the two types of reagents, the second reagent can be substituted by a dry powdered reagent. After the dry powdered reagent is coated onto the internal wall of the capillary portion 303 of the mixing interval 301, the first reagent is dropped into the first inlet 304, and flows through the mixing interval 301 to mix with the dry powdered reagent and start reacting, and the mixed liquids will be driven by the capillary force to continue passing the mixing interval 301 and flow towards the outlet 306 to facilitate other following tests.

In another embodiment, the middle layer 32 can be a double-sided adhesive tape. In practical usage, the middle layer 32 can be a hydrophilic JSR photoresist material or polymethylmethacrylate (PMMA) photoresist material. If the aforementioned two types of hydrophilic photoresist materials are used to make the middle layer 32, the hydrophilic photoresist material can be fixed to the substrate layer 31, and the exposure and development methods can be used to make the microchannel 321. Since the substrate layer 31, the middle layer 32 and the cap layer 33 are made of a hydrophilic material, the capillary force of the microfluidic channel 300 is increased to improve the efficiency of transporting the liquids. Also, since the photoresist materials can be bonded and fixed to the substrate layer 31 and the cap layer 33 (both made of glass) at a lower temperature, a simple and quick manufacturing process can be provided.

In summary, the hydrophilic materials can be used for making the substrate layer 31 and the cap layer 33, while the double-sided adhesive tape or hydrophilic photoresist material can be used for making the middle layer 32 to produce the biomedical chip for blood coagulation tests 3, and the capillary force of the microfluidic channel 300 can drive highly viscous blood to flow automatically. By using the design of the first inlet 304 and the second inlet 305 disposed with an interval apart from each other and along the lengthwise direction of the microfluidic channel 300 which is interconnected to the mixing interval 301 of the second inlet 305, the two types of reagents can be mixed and transported effectively for performing the blood coagulation test. In addition, the reagent added into the mixing interval 301 can be a powdered reagent or a liquid reagent. Therefore, the biomedical chip for blood coagulation tests 3 of the present invention can be used extensively in different areas, particularly in applications for medical treatments in intensive care units, emergency rooms and operation rooms that require an immediate blood coagulation test to improve the medical treatment quality, and the invention is also applicable for the general public for taking a blood coagulation test. Obviously, the present invention has commercial value and potential.

In addition, the hydrophilic substrate layer 31, the middle layer 32 and the cap layer 33 can maintain the hydrophilic property permanently during the process of usage, without worrying about the chip from resuming its hydrophobic property after being modified to the hydrophilic surface. In addition, the double-sided adhesive tape or the hydrophilic photoresist material such as JSR and PMMA is used for the design of the middle layer 32, such that the substrate layer 31, the middle layer 32 and the cap layer 33 can be bonded at a lower temperature to facilitate the manufacture, so as to achieve the objectives of the present invention.

While the invention has been described by means of specific embodiments, numerous modifications and variations could be made thereto by those skilled in the art without departing from the scope and spirit of the invention set forth in the claims.

What is claimed is:

1. A biomedical chip for blood coagulation tests, comprising:
a substrate layer made of a hydrophilic material, a middle layer, and a cap layer made of a hydrophilic material, sequentially engaged and stacked from bottom to top with each other, wherein the substrate layer, the middle layer and the cap layer define a microfluidic channel formed at the cap layer, having a first inlet and an outlet at two opposite ends of the microfluidic channel respectively; a second inlet being disposed proximate to the first inlet and interconnected to the microfluidic channel; and a mixing interval expanded radially outward and interconnected to the second inlet and the externally expanded mixing interval having a diameter greater than the diameter of the second inlet, wherein an interconnect portion is disposed at the bottom of the second inlet; and a capillary portion is disposed between the substrate layer and the cap layer, and interconnected to the interconnect portion and disposed around the periphery thereof, wherein the microfluidic channel's internal diameter is small enough to produce a capillary force to drive blood in the first inlet and a reagent in the second inlet to flow along a lengthwise direction, such that the blood is driven automatically to pass through the capillary portion to absorb the reagent in the interconnect portion and flow towards the outlet.

2. The biomedical chip for blood coagulation tests of claim 1, wherein the substrate layer and the cap layer are made of glass.

3. The biomedical chip for blood coagulation tests of claim 2, wherein the middle layer is made of a photoresist material with a hydrophilic property.

4. The biomedical chip for blood coagulation tests of claim 3, wherein the middle layer is made of a material selected from the collection of a JSR photoresist material and a polymethylmethacrylate (PMMA) photoresist material.

5. The biomedical chip for blood coagulation tests of claim 4, wherein the middle layer includes a slender microchannel penetrated through the middle layer, and defined by the middle layer, the substrate layer and the cap layer, and the microchannel has an externally expanded section expanded radially outward to define the mixing interval.

6. The biomedical chip for blood coagulation tests of claim 2, wherein the middle layer is a double-sided adhesive tape.

7. The biomedical chip for blood coagulation tests of claim 5, wherein the middle layer includes a slender microchannel penetrated through the middle layer, and defined by the middle layer, the substrate layer and the cap layer, and the microchannel has an externally expanded section expanded radially outward to define the mixing interval.

8. A method of manufacturing a biomedical chip for blood coagulation tests, comprising the steps of:
(a) attaching and fixing a middle layer onto the top of a hydrophilic substrate layer;
(b) forming a slender penetrating microchannel at the middle layer, wherein the microchannel has an externally expanded section radially expanded outward;
(c) forming a first inlet hole, a second inlet hole and a outlet hole with an interval apart from each other and passed through the top side of the hydrophilic cap layer by a laser manufacturing method, and the second inlet hole having a diameter smaller than the diameter of the externally expanded section; and
(d) stacking and fixing the cap layer produced in Step (c) onto the top side of the middle layer to cover the microchannel, such that the cap layer, the substrate layer and the middle layer define an internal diameter of the microchannel which is small enough to produce a capillary force for driving the blood to flow in the microfluidic channel automatically, and the first filling hole and the discharging hole are interconnected to two opposite ends of the microfluidic channel respectively, and the second inlet hole and the externally expanded section are interconnected correspondingly.

9. The method of manufacturing a biomedical chip for blood coagulation tests as recited in claim 8, wherein the middle layer as described in Step (a) is a double-sided adhesive tape adhered and fixed directly to the top side of the substrate layer, and the microchannel is manufactured and formed on the middle layer by a laser manufacturing method as described in Step (b), and the cap layer is stacked and adhered with the top of the middle layer directly as described in Step (d).

10. The method of manufacturing a biomedical chip for blood coagulation tests as recited in claim 8, wherein the middle layer as described in Step (a) is made of a hydrophilic photoresist material, and the microchannel is formed on the middle layer as described in Step (b) through an exposure and development method.

11. The method of manufacturing a biomedical chip for blood coagulation tests as recited in claim 10, wherein the middle layer is made of a material selected from the collection of a JSR photoresist material and a polymethylmethacrylate (PMMA) photoresist material.

12. A use of the biomedical chip for blood coagulation tests as recited in claim 1 to drive and mix two types of reagents, and the use comprising the steps of:
(a) dropping a first reagent into the first inlet, and using the capillary force produced by the microfluidic channel to drive the first reagent to flow automatically; and
(b) adding a second reagent to be mixed into the interconnect portion from the second inlet after the first reagent is passed along the capillary portion of the mixing interval, bypassed through the interconnect portion of the mixing interval, and passed through the mixing interval, such that after the second reagent is sucked into the capillary portion by the capillary force of the capillary portion and mixed with the first reagent, the first and second reagents are mixed and passed through the mixing interval.

13. A use of the biomedical chip for blood coagulation tests as recited in claim 1 to drive and mix two types of reagents, and the use comprising the steps of:
(a) coating a dry powdered second reagent onto an internal wall of the mixing interval; and
(b) dropping a liquid first reagent into the first inlet, and using the capillary force produced by the microfluidic channel to drive the first reagent to flow automatically, such that the first reagent passing through the capillary portion gradually carries away the dry powdered second reagent in the mixing space, and after the second reagent is mixed with the first reagent, the first and second reagents are passed through the mixing interval.
